(19) United States
(12) Patent Application Publication
(10) Pub. No.: US 2026/0144907 A1
(43) Pub. Date: May 28, 2026

(54) READER DEVICE, STERILIZATION INDICATOR SYSTEM, AND METHOD

(71) Applicant: Solventum Intellectual Properties Company, Maplewood, MN (US)

(72) Inventors: Benjamin M. Wilke, Shoreview, MN (US); G. Marco Bommarito, Stillwater, MN (US); Andrew J. Ma, Woodbury, MN (US); Anthony J. Jennen, Vadnais Heights, MN (US); Anthony Hernandez, San Antonio, TX (US)

(73) Assignee: Solventum Intellectual Properties Company, Maplewood, MN (US)

(21) Appl. No.: 19/121,602

(22) PCT Filed: Oct. 9, 2023
(86) PCT No.: PCT/IB2023/060132

§ 371 Date: Apr. 16, 2025

Related U.S. Application Data
(60) Provisional application No. 63/379,778, filed on Oct. 17, 2022.

(51) Int. Cl. A61L 2/28; A61L 2/07; A61L 103/15; G01D 5/16; G01N 27/20

(52) U.S. Cl. CPC A61L 2/28; G01D 5/16; G01N 27/20; A61L 2/07; A61L 2103/15; A61L 2202/14

(57) ABSTRACT

A reader device includes pluralities of first and second contacts and a controller. The first contacts are configured to electrically contact corresponding one or more conductive pads of the sensor device. The first contacts include a first open state in which the first contacts are electrically disconnected and a first closed state in which the first contacts are electrically connected. The second contacts are spaced apart from the first contacts. The second contacts are configured to electrically contact corresponding one or more conductive pads of the sensor device. The second contacts include a second open state in which the second contacts are electrically disconnected and a second closed state in which the second contacts are electrically connected. The controller is configured to determine an error condition if the first contacts are in the first open state and/or the second contacts are in the second open state.

### TECHNICAL FIELD

The present disclosure relates to a reader device for use with a sensor device, a sterilization indicator system including the reader device and the sensor device, and a method.

### BACKGROUND

Sterilization of equipment (e.g., medical and hospital equipment) may not be effective until a steam sterilant has been in contact with all surfaces of the equipment being sterilized in a proper combination of time, temperature, and steam quality. In steam sterilizers, such as pre-vacuum steam sterilizers and gravity displacement steam sterilizers, the process of sterilization is conducted in three main phases. In the first phase, air is removed, including air trapped within any porous materials being processed. The first phase is therefore an air removal phase. The second phase is a sterilizing phase, in which a load (i.e., the equipment being sterilized) is subjected to steam under pressure for a recognized, predetermined combination of time and temperature to effect sterilization. The third phase is a drying phase in which condensation formed during the first two phases is removed by evacuating the steam sterilizer.

Any air that is not removed from the steam sterilizer during the air removal phase of the cycle or which leaks into the steam sterilizer during a sub atmospheric pressure stage due to, for example, faulty gaskets, valves, or seals, may form air pockets within any porous materials present. Such air pockets may create a barrier to steam penetration, thereby preventing adequate sterilizing conditions being achieved for all surfaces of the load during the sterilizing phase. For example, these air pockets may prevent the steam from reaching interior layers of materials, such as hospital linens or fabrics. In some other examples, these air pockets may prevent the steam from penetrating hollow spaces of tubes, catheters, syringe needles, and the like. Further, non-condensable gas (generally air) present within the steam sterilizer is a poor sterilant and may decrease sterilization efficacy. Therefore, the presence of air pockets and/or non-condensable gas may affect a steam quality of the steam sterilant. As a result, proper sterilization may not occur due to reduced steam quality.

Various sterilization indicators are widely used in sterilization monitoring to ensure the sterilization process has been completed correctly. Failed or insufficient sterilization cycles may put patients in huge risk due to the potential cross-contaminations from the reprocessed equipment. Traditional sterilization indicators include chemical and biological sterilization indicators. A Bowie-Dick test pack is a type of chemical sterilization indicator designed to detect air leak or insufficient air removal in the steam sterilizer.

### SUMMARY

In a first aspect, the present disclosure provides a reader device for use with a sensor device. The sensor device has a plurality of conductive pads. The reader device includes a body defining a slot configured to at least partially receive the sensor device therein. The reader device further includes a plurality of first conductive contacts disposed in the slot and adjacent to each other. The plurality of first conductive contacts is configured to electrically contact corresponding one or more conductive pads from the plurality of conductive pads of the sensor device upon insertion of the sensor device within the slot. The plurality of first conductive contacts includes a first open state and a first closed state. In the first open state, the first conductive contacts are electrically disconnected from each other. In the first closed state, the first conductive contacts are electrically connected to each other. The reader device further includes a plurality of second conductive contacts disposed in the slot and adjacent to each other. The plurality of second conductive contacts is spaced apart from the plurality of first conductive contacts. The plurality of second conductive contacts is configured to electrically contact corresponding one or more conductive pads from the plurality of conductive pads of the sensor device upon insertion of the sensor device within the slot. The plurality of second conductive contacts includes a second open state and a second closed state. In the second open state, the second conductive contacts are electrically disconnected from each other. In the second closed state, the second conductive contacts are electrically connected to each other. The reader device further includes a controller communicably coupled to at least one of the plurality of first conductive contacts and at least one the plurality of second conductive contacts. The controller is configured to determine, upon insertion of the sensor device within the slot, an error condition if the plurality of first conductive contacts is in the first open state and/or the plurality of second conductive contacts is in the second open state. In the error condition, the sensor device is in an incorrect position with respect to the reader device. The controller is further configured to generate an error signal upon determining the error condition.

In a second aspect, the present disclosure provides a sterilization indicator system. The sterilization indicator system includes a sensor device including a plurality of conductive pads. The sterilization indicator system further includes the reader device of the first aspect. Upon insertion of the sensor device within the slot, the plurality of first conductive contacts is configured to electrically contact corresponding one or more first conductive pads from the plurality of conductive pads of the sensor device and the plurality of second conductive contacts is configured to electrically contact corresponding one or more second conductive pads from the plurality of conductive pads of the sensor device.

In a third aspect, the present disclosure provides a method. The method includes inserting a sensor device having a plurality of conductive pads within a slot of a reader device. The reader device includes a plurality of first conductive contacts disposed in the slot and adjacent to each other. The plurality of first conductive contacts is configured to electrically contact corresponding one or more conductive pads from the plurality of conductive pads of the sensor device upon insertion of the sensor device within the slot. The plurality of first conductive contacts includes a first open state and a first closed state. In the first open state, the first conductive contacts are electrically disconnected from each other. In the first closed state, the first conductive contacts are electrically connected to each other. The reader device further includes a plurality of second conductive contacts disposed in the slot and adjacent to each other. The plurality of second conductive contacts is spaced apart from the plurality of first conductive contacts. The plurality of second conductive contacts is configured to electrically contact corresponding one or more conductive pads from the plurality of conductive pads of the sensor device upon insertion of the sensor device within the slot. The plurality of second conductive contacts includes a second open state and a second closed state. In the second open state, the second conductive contacts are electrically disconnected from each other. In the second closed state, the second conductive contacts are electrically connected to each other. The method further includes determining an error condition if the plurality of first conductive contacts of the reader device is in the first open state and/or the plurality of second conductive contacts of the reader device is in the second open state. In the error condition, the sensor device is in an incorrect position with respect to the reader device. The method further includes generating an error signal upon determining the error condition.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 shows a schematic view of a sterilization indicator system, according to an embodiment of the present disclosure;

FIG. 2 shows a schematic perspective view of a reader device of the sterilization indicator system, according to an embodiment of the present disclosure;

FIG. 3A shows a schematic sectional view of the reader device, according to an embodiment of the present disclosure;

FIG. 3B shows a magnified schematic sectional view of a portion of the reader device, according to an embodiment of the present disclosure;

FIG. 4A shows a schematic block diagram of the sterilization indicator system in an error condition, according to an embodiment of the present disclosure;

FIG. 4B shows a schematic block diagram of the sterilization indicator system in a correct condition, according to an embodiment of the present disclosure;

FIG. 5A shows a schematic view of the sterilization indicator system with a sensor device in an unused condition, according to another embodiment of the present disclosure;

FIG. 5B shows a schematic view of the sterilization indicator system with the sensor device in a used condition, according to an embodiment of the present disclosure;

FIG. 6A shows a schematic view of the sensor device in the unused condition and an accessory, according to an embodiment of the present disclosure;

FIG. 6B shows a schematic view of the sensor device being used with the accessory, according to an embodiment of the present disclosure;

FIG. 6C shows a schematic view of the sensor device in the used condition, according to an embodiment of the present disclosure;

FIG. 7A is a schematic perspective view of the sterilization indicator system in the correct condition, according to an embodiment of the present disclosure;

FIG. 7B is a schematic perspective view of the sterilization indicator system in the correct condition, according to an embodiment of the present disclosure;

FIG. 7C is a schematic perspective view of the sterilization indicator system in the error condition, according to an embodiment of the present disclosure; and

FIG. 8 is a flowchart illustrating a method, according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As used herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, “at least one of A and B” should be understood to mean “only A, only B, or both A and B”.

As used herein, the term “controller” refers to a computing device that couples to one or more other devices/circuits, e.g., switching circuits, etc., and which may be configured to communicate with, e.g., to control, such devices/circuits. The controller may include any device that performs logic operations. A controller may include a general processor, a central processing unit, an application specific integrated circuit (ASIC), a digital signal processor, a field programmable gate array (FPGA), a digital circuit, an analog circuit, a microcontroller, any other type of controller, or any combination thereof.

As used herein, the term “electrically connected” refers to direct coupling between components and/or indirect coupling between components via one or more intervening electric components, such that electric current can be passed between the two components. As an example of indirect coupling, two components can be referred to as being electrically coupled, even though they may have an intervening electric component between them which still allows electric current to pass from one component to the other component. Such intervening components may include, but are not limited to, wires, traces on a circuit board, and/or another electrically conductive medium/component.

As used herein, the terms “conductive contacts” and “conductive pads” refer to electrical connections that interconnect analog components and circuitry to other components and wires in a circuit. The electrical connections may include wires, traces on a circuit board, and/or another electrically conductive medium/component.

As used herein, the term “communicably coupled” refers to direct coupling between components and/or indirect coupling between components via one or more intervening components. Such components and intervening components may include, but are not limited to, junctions, communication paths, components, circuit elements, circuits, functional blocks, and/or devices. As an example of indirect coupling, a signal conveyed from a first component to a second component may be modified by one or more intervening components by modifying the form, nature, or format of information in a signal, while one or more elements of the information in the signal are nevertheless conveyed in a manner than can be recognized by the second component.

As used herein, the term “signal” includes, but is not limited to, one or more electrical signals, optical signals, electromagnetic signals, analog and/or digital signals, one or more computer instructions, a bit and/or bit stream, or the like.

In steam sterilizers, such as pre-vacuum steam sterilizers and gravity displacement steam sterilizers, the process of sterilization is conducted in three main phases. In the first phase, air is removed, including air trapped within any porous materials being processed. The first phase is therefore an air removal phase. The second phase is a sterilizing phase, in which a load (i.e., equipment being sterilized) is subjected to steam under pressure for a recognized, predetermined combination of time and temperature to effect sterilization. The third phase is a drying phase in which condensation formed during the first two phases is removed by evacuating the sterilizer. Any air that is not removed from the steam sterilizer during the air removal phase of the cycle or which leaks into the sterilizer during a sub atmospheric pressure stage due to, for example, faulty gaskets, valves, or seals, may form air pockets within any porous materials present. Such air pockets may create a barrier to steam penetration, thereby preventing adequate sterilizing conditions being achieved for all surfaces of the load during the sterilizing phase. Further, non-condensable gas (generally air) present within the steam sterilizer is a poor sterilant and may decrease sterilization efficacy. Therefore, the presence of air pockets and/or non-condensable gas may affect a steam quality of the steam sterilant. As a result, proper sterilization may not occur due to reduced steam quality.

Various sterilization indicators are widely used in sterilization monitoring to ensure the sterilization process has been completed correctly. Failed or insufficient sterilization cycles may put patients in huge risk due to the potential cross-contaminations from the reprocessed equipment. Traditional sterilization indicators include chemical and biological sterilization indicators. A chemical sterilization indicator may be designed to detect air leak or insufficient air removal in the steam sterilizer.

In some cases, the sterilization indicator may change one or more electrical parameters based on the efficacy of the sterilization process. Further, a reader may measure the change in the one or more electrical parameters and generate a pass result or a fail result based on the measurement. However, in some cases, the measurement by the reader may be affected due to a malfunction of the reader/sterilization indicator or an improper insertion of the sterilization indicator in the reader. For example, the sterilization indicator may have a large resistance value in case of sufficient air removal in the steam sterilizer. The reader may further measure the large resistance value and generate the pass result. However, the reader may also measure a large resistance value in case of the malfunction of the reader/sterilization indicator or the improper insertion of the sterilization indicator in the reader, and may falsely generate the pass result. This may put the patients in a huge risk.

Therefore, a suitable solution may be required which may be able to determine if the sterilization indicator actually provides the pass result, or if there is a malfunction or an improper insertion of the sterilization indicator in the reader.

The present disclosure relates to a reader device for use with a sensor device, a sterilization indicator system including the reader device and the sensor device, and a method. The sensor device has a plurality of conductive pads.

The reader device includes a body defining a slot configured to at least partially receive the sensor device therein. The reader device further includes a plurality of first conductive contacts disposed in the slot and adjacent to each other. The plurality of first conductive contacts is configured to electrically contact corresponding one or more conductive pads from the plurality of conductive pads of the sensor device upon insertion of the sensor device within the slot. The plurality of first conductive contacts includes a first open state and a first closed state. In the first open state, the first conductive contacts are electrically disconnected from each other. In the first closed state, the first conductive contacts are electrically connected to each other. The reader device further includes a plurality of second conductive contacts disposed in the slot and adjacent to each other. The plurality of second conductive contacts is spaced apart from the plurality of first conductive contacts. The plurality of second conductive contacts is configured to electrically contact corresponding one or more conductive pads from the plurality of conductive pads of the sensor device upon insertion of the sensor device within the slot. The plurality of second conductive contacts includes a second open state and a second closed state. In the second open state, the second conductive contacts are electrically disconnected from each other. In the second closed state, the second conductive contacts are electrically connected to each other. The reader device further includes a controller communicably coupled to at least one of the plurality of first conductive contacts and at least one the plurality of second conductive contacts. The controller is configured to determine, upon insertion of the sensor device within the slot, an error condition if the plurality of first conductive contacts is in the first open state and/or the plurality of second conductive contacts is in the second open state. In the error condition, the sensor device is in an incorrect position with respect to the reader device. The controller is further configured to generate an error signal upon determining the error condition.

The reader device of the present disclosure may be able to determine the error condition when the sensor device is in the incorrect position with respect to the reader device. The reader device may perform a check before measurement of one or more electrical parameters across the plurality of conductive pads of the sensor device. This may prevent the reader device to erroneously generate a pass result or a failure result when the sensor device is in the incorrect position with respect to the reader device. Thus, the reader device of the present invention may be robust and reliable in contrast to a conventional reader which directly measures the one or more electrical parameters upon insertion of the sterilization indicator and therefore may falsely generate the pass result.

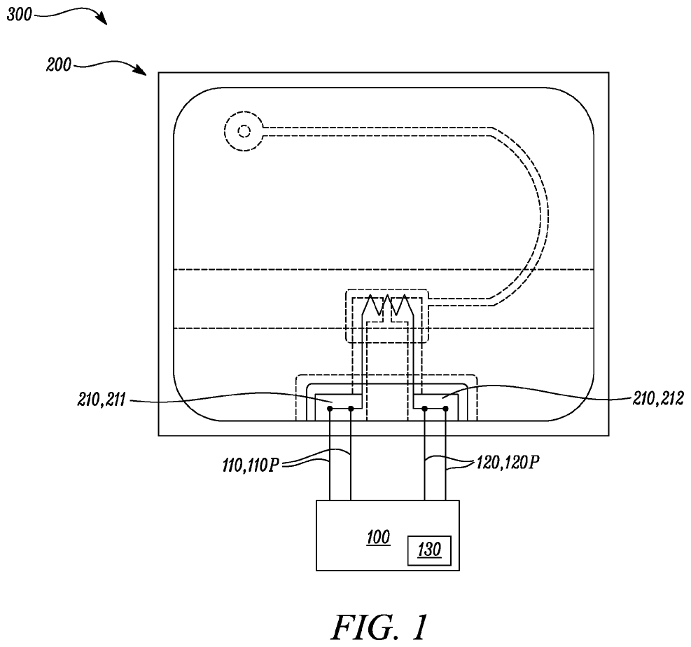

Referring now to figures, FIG. 1 is a schematic view of a sterilization indicator system 300, according to an embodiment of the present disclosure.

The sterilization indicator system 300 includes a reader device 100. The reader device 100 includes a plurality of first conductive contacts 110. The reader device 100 further includes a plurality of second conductive contacts 120. The reader device 100 further includes a controller 130. The sterilization indicator system 300 further includes a sensor device 200. The sensor device 200 includes a plurality of conductive pads 210.

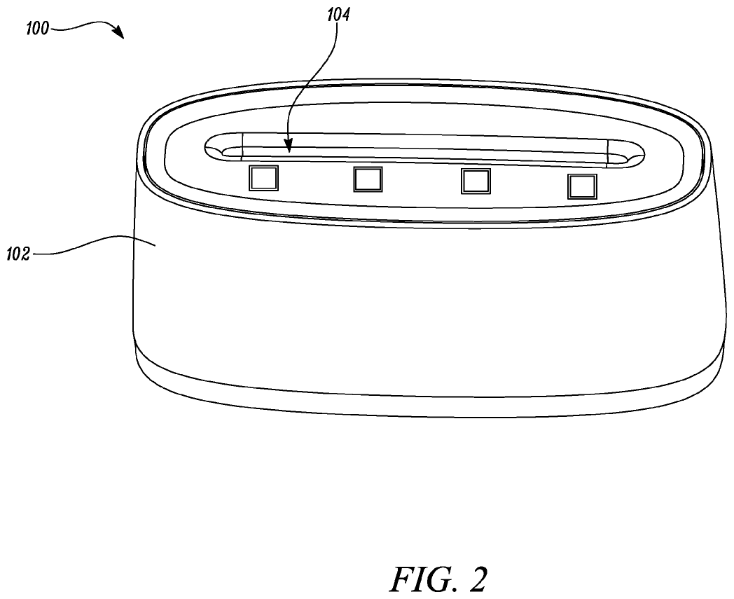

FIG. 2 is a schematic perspective view of the reader device 100 for use with the sensor device 200 shown in FIG. 1, according to an embodiment of the present disclosure. The reader device 100 includes a body 102. The body 102 defines a slot 104 therein. Referring to FIGS. 1 and 2, the slot 104 is configured to at least partially receive the sensor device 200 therein.

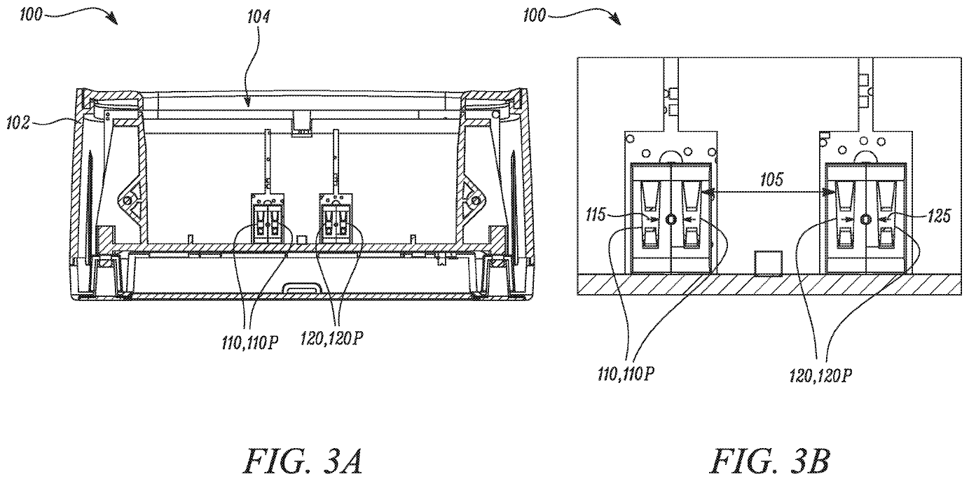

FIG. 3A is a schematic sectional view of the reader device 100, according to an embodiment of the present disclosure. FIG. 3B is a magnified schematic sectional view of a portion of the reader device 100, according to an embodiment of the present disclosure.

Referring to FIGS. 1 to 3B, the plurality of first conductive contacts 110 is disposed in the slot 104. Further, the plurality of first conductive contacts 110 is disposed adjacent to each other. Similarly, the plurality of second conductive contacts 120 is disposed in the slot 104. Further, the plurality of second conductive contacts 120 is disposed adjacent to each other. The plurality of second conductive contacts 120 is spaced apart from the plurality of first conductive contacts 110. In some embodiments, the plurality of first conductive contacts 110 and the plurality of second conductive contacts 120 may include position spring compression contacts.

The plurality of first conductive contacts 110 is configured to electrically contact corresponding one or more conductive pads (interchangeably referred to herein as “one or more first conductive pads”) from the plurality of conductive pads 210 of the sensor device 200 upon insertion of the sensor device 200 within the slot 104. Similarly, the plurality of second conductive contacts 120 is configured to electrically contact corresponding one or more conductive pads 210 (interchangeably referred to herein as “one or more second conductive pads”) from the plurality of conductive pads 210 of the sensor device 200 upon insertion of the sensor device 200 within the slot 104. In other words, upon insertion of the sensor device 200 within the slot 104, the plurality of first conductive contacts 110 is configured to electrically contact corresponding one or more first conductive pads from the plurality of conductive pads 210 of the sensor device 200 and the plurality of second conductive contacts 120 is configured to electrically contact corresponding one or more second conductive pads from the plurality of conductive pads 210 of the sensor device 200. The plurality of conductive pads 210 of the sensor device 200 may serve as designated conductive surface areas for electrical contact between the sensor device 200 and the reader device 100.

As is illustrated in the embodiment of FIGS. 1 to 3B, the plurality of first conductive contacts 110 is spaced apart from each other by a first distance 115. Specifically, two adjacent first conductive contacts 110 are spaced apart from each other by the first distance 115. Similarly, the plurality of second conductive contacts 120 is spaced apart from each other by a second distance 125. Specifically, two adjacent second conductive contacts 120 are spaced apart from each other by the second distance 125. In some embodiments, the first distance 115 and the second distance 125 may be substantially equal to each other. In some embodiments, the first distance 115 and the second distance 125 may not be equal.

In some embodiments, each of the first distance and the second distance is less than or equal to about 5 millimeters (mm) and greater than or equal to about 1 mm. In some embodiments, each of the first distance and the second distance is equal to about 4 mm.

Further, in some embodiments, the plurality of second conductive contacts 120 is spaced apart from the plurality of first conductive contacts 110 by a minimum distance 105. As is apparent from FIG. 3B, the minimum distance 105 is greater than each of the first distance 115 and the second distance 125. In some embodiments, the minimum distance 105 is greater than each of the first distance 115 and the second distance 125 by a factor of at least 5, at least 6, at least 8, or at least 10.

In the illustrated embodiments of FIGS. 1 to 3B, the plurality of first conductive contacts 110 includes a pair of first conductive contacts 110P spaced apart from each other. Specifically, the pair of first conductive contacts 110P are spaced apart from each other by the first distance 115. Similarly, the plurality of second conductive contacts 120 includes a pair of second conductive contacts 120P spaced apart from each other. Specifically, the pair of second conductive contacts 120P are spaced apart from each other by the second distance 125.

The pair of first conductive contacts 110P is configured to electrically contact a first conductive pad 211 from the plurality of conductive pads 210 of the sensor device 200 upon insertion of the sensor device 200 within the slot 104. Specifically, upon insertion of the sensor device 200 within the slot 104, the pair of first conducive contacts 110P is configured to at least partially receive the first conductive pad 211 therebetween, such that the first conductive pad 211 contacts each of the pair of first conducive contacts 110P. Further, the pair of second conductive contacts 120P is configured to electrically contact a second conductive pad 212 from the plurality of conductive pads 210 of the sensor device 200 upon insertion of the sensor device 200 within the slot 104. Specifically, upon insertion of the sensor device 200 within the slot 104, the pair of second conducive contacts 120P is configured to at least partially receive the second conductive pad 212 therebetween, such that the second conductive pad 212 contacts each of the pair of second conductive contacts 120P.

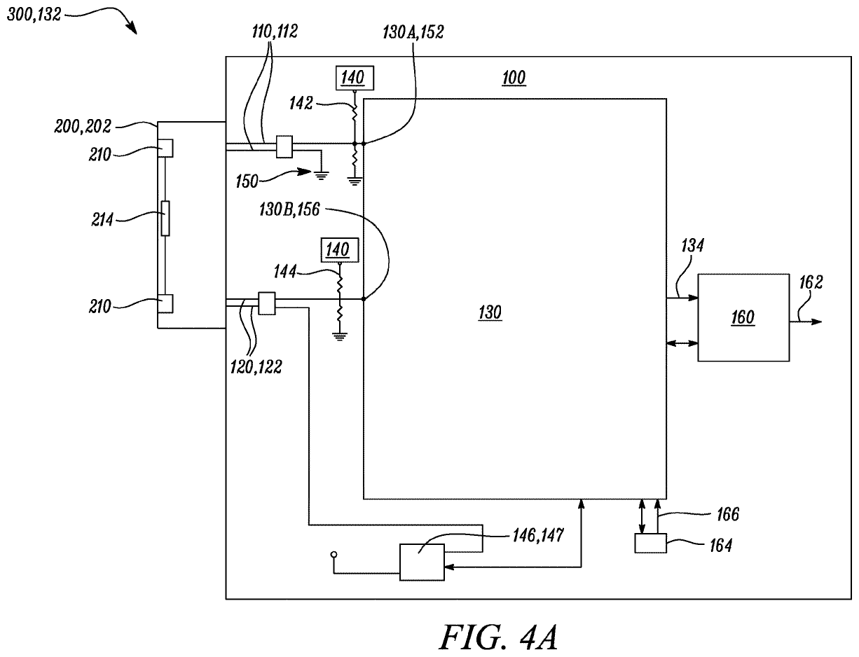

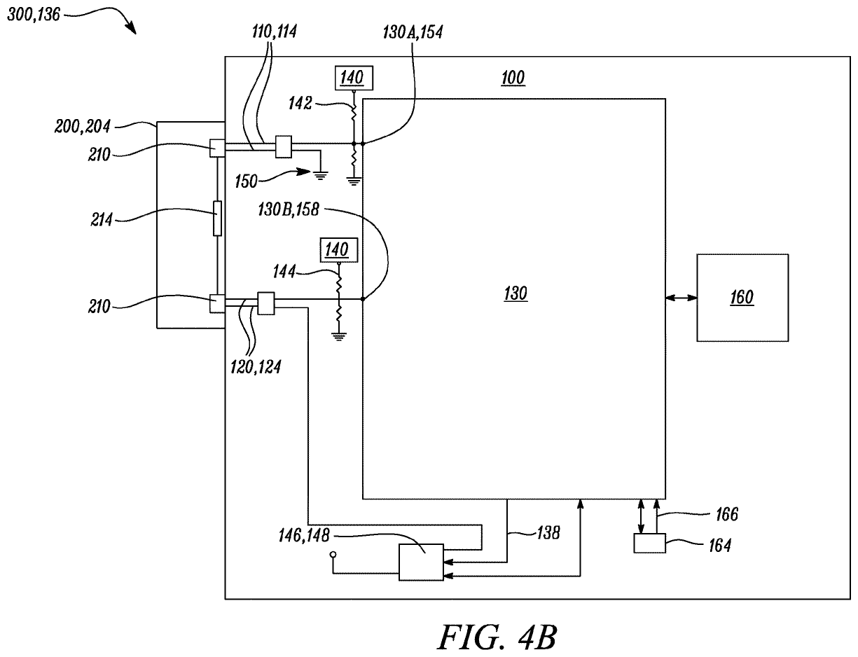

FIG. 4A is a schematic block diagram of the sterilization indicator system 300 in an error condition 132, according to an embodiment of the present disclosure. In the error condition 132, the sensor device 200 is in an incorrect position 202 with respect to the reader device 100.

FIG. 4B is a schematic block diagram of the sterilization indicator system 300 in a correct condition 136, according to an embodiment of the present disclosure. In the correct condition 136, the sensor device 200 is in a correct position 204 with respect to the reader device 100.

Referring to FIGS. 4A and 4B, the plurality of first conductive contacts 110 includes a first open state 112 (shown in FIG. 4A) and a first closed state 114 (shown in FIG. 4B). In the first open state 112, the first conductive contacts 110 are electrically disconnected from each other. In the first closed state 114, the first conductive contacts 110 are electrically connected to each other.

Further, the plurality of second conductive contacts 120 includes a second open state 122 (shown in FIG. 4A) and a second closed state 124 (shown in FIG. 4B). In the second open state 122, the second conductive contacts 120 are electrically disconnected from each other. In the second closed state 124, the second conductive contacts 120 are electrically connected to each other.

The controller 130 is communicably coupled to at least one of the plurality of first conductive contacts 110 and at least one the plurality of second conductive contacts 120.

In some embodiments, the reader device 100 further includes a sensor 164 communicably coupled to the controller 130. The sensor 164 is configured to generate an insertion signal 166 upon sensing the insertion of the sensor device 200 within the slot 104 (shown in FIG. 2). In some embodiments, the controller 130 is further configured to determine that the sensor device 200 has been inserted within the slot 104 upon receiving the insertion signal 166 from the sensor 164. In some embodiments, the sensor 164 is an infrared sensor.

The controller 130 is configured to determine, upon insertion of the sensor device 200 within the slot 104, the error condition 132 (shown in FIG. 4A) if the plurality of first conductive contacts 110 is in the first open state 112 and/or the plurality of second conductive contacts 120 is in the second open state 122. The controller 130 is further configured to generate an error signal 134 upon determining the error condition 132.

In some embodiments, the reader device 100 further includes an alert circuit 160 (shown in FIG. 4A) communicably coupled to the controller 130. The alert circuit 160 is configured to generate an error alert 162 upon receiving the error signal 134 from the controller 130. In some embodiments, the error alert 162 is at least one of a visual alert, an audio alert, and a haptic alert.

In some embodiments, the controller 130 is further configured to determine, upon insertion of the sensor device 200 within the slot 104, the correct condition 136 (shown in FIG. 4B) if the plurality of first conductive contacts 110 is in the first closed state 114 and the plurality of second conductive contacts 120 is in the second closed state 124. In some embodiments, the controller 130 is further configured to generate a correct signal 138 upon determining the correct condition 136. In some embodiments, the alert circuit 160 may be configured to generate a correct alert (not shown) upon receiving the correct signal 138 from the controller 130. In some embodiments, the correct alert is at least one of a visual alert, an audio alert, and a haptic alert.

In some embodiments, the controller 130 is further configured to measure at least one electrical parameter 214 across the plurality of conductive pads 210 upon determining the correct condition 136. The at least one electrical parameter 214 is indicative of an efficacy of a sterilization process. In some embodiments, the at least one electrical parameter 214 includes at least one of a voltage, a current flow, a resistance, and an impedance across the plurality of conductive pads 210.

Table 1 summarizes different positions of the sensor device 200 upon insertion of the sensor device 200 within the slot 104, different states of the plurality of first conductive contacts 110 and the plurality of second conductive contacts 120 in the different positions of the sensor device 200, different conditions determined by the controller 130 based on the different states of the plurality of first conductive contacts 110 and the plurality of second conductive contacts 120, and different signals generated by the controller 130 upon determining the different conditions.

TABLE 1

| Sensor | First | Second | | |
|---|---|---|---|---|
| Device | Conductive | Conductive | | |
| Position | Contacts | Contacts | Condition | Signals |
| Incorrect | First Open | Second Open | Error | Error |
| Position | State | State | Condition | Signal |
| Incorrect | First Open | Second Closed | Error | Error |
| Position | State | State | Condition | Signal |
| Incorrect | First Closed | Second Open | Error | Error |
| Position | State | State | Condition | Signal |
| Correct | First Closed | Second Closed | Correct | Correct |
| Position | State | State | Condition | Signal |

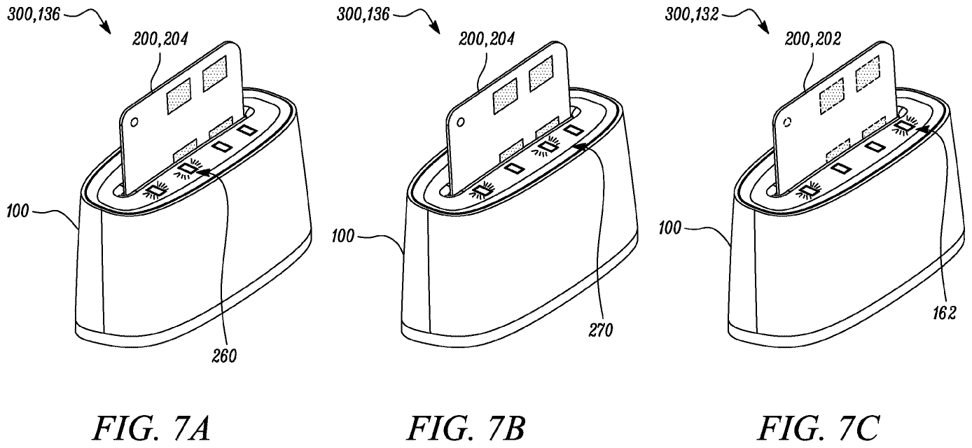

Therefore, the reader device 100 may be able to determine the error condition 132 when the sensor device 200 is in the incorrect position 202 with respect to the reader device 100 or the correct condition 136 when the sensor device 200 is in the correct position 204 with respect to the reader device 100. The reader device 100 may therefore perform an electrical continuity check via the plurality of first conductive contacts 110 and the corresponding one or more conductive pads 210 and the plurality of second conductive contacts 120 and the corresponding one or more conductive pads 210 before measurement of the at least one electrical parameter 214 across the plurality of conductive pads 210 of the sensor device 200. Specifically, when the sensor device 200 is in the incorrect position 202 with respect to the reader device 100, there may be no electrical continuity between the plurality of first conductive contacts 110 and the corresponding one or more conductive pads 210 and/or the plurality of second conductive contacts 120 and the corresponding one or more conductive pads 210. The reader device 100 may therefore only measure the at least one electrical parameter 214 across the plurality of conductive pads 210 of the sensor device 200, when the sensor device 200 is in the correct position 204 with respect to the reader device 100. Since, in the correct position 204, the plurality of first conductive contacts 110 contacts the corresponding one or more conductive pads 210 and the plurality of second conductive contacts 120 contacts the corresponding one or more conductive pads 210, there is an electrical continuity between the plurality of first conductive contacts 110 and the corresponding one or more conductive pads 210 and the plurality of second conductive contacts 120 and the corresponding one or more conductive pads 210. Therefore, the reader device 100 may only measure the at least one electrical parameter 214 across the plurality of conductive pads 210 of the sensor device 200, when there is an electrical continuity between the plurality of first conductive contacts 110 and the corresponding one or more conductive pads 210 and the plurality of second conductive contacts 120 and the corresponding one or more conductive pads 210. This may prevent the reader device 100 to erroneously generate a pass alert 260 (shown in FIG. 7A) or a failure alert 270 (shown in FIG. 7B) when the sensor device 200 is in the incorrect position 202 with respect to the reader device 100. Thus, the reader device 100 may be robust and reliable in contrast to a conventional reader which directly measures one or more electrical parameters upon insertion of a sterilization indicator and therefore may falsely generate the pass alert 260.

In some embodiments, the reader device 100 further includes a voltage source 140, a first pullup resistor 142, a second pullup resistor 144, and a supply circuit 146. The supply circuit 146 is electrically connected to the voltage source 140. Further, the controller 130 is communicably coupled to the supply circuit 146. The controller 130 includes a first pin 130A and a second pin 130B. In some embodiments, the supply circuit 146 may be electrically connected to a power source (not shown).

In some embodiments, one of the pair of first conductive contacts 110P (shown in FIGS. 3A, 3B) (i.e., the plurality of first conductive contacts 110) is electrically connected to the first pin 130A of the controller 130 and the voltage source 140. The first pullup resistor 142 is electrically disposed between the one of the pair of first conductive contacts 110P and the voltage source 140. In some embodiments, the other of the pair of first conductive contacts 110P is electrically connected to electrical ground 150.

In some embodiments, one of the pair of second conductive contacts 120P (shown in FIGS. 3A, 3B) (i.e., the plurality of first conductive contacts 120) is electrically connected to the second pin 130B of the controller 130 and the voltage source 140. The second pullup resistor 144 is electrically disposed between the one of the pair of second conductive contacts 120P and the voltage source 140. In some embodiments, the other of the pair of second conductive contacts 120P is electrically connected to the supply circuit 146. The supply circuit 146 provides a first voltage 147 equal to about 0 volt (V) when the sensor device 200 is inserted within the slot 104 (shown in FIG. 2).

In the first open state 112 (shown in FIG. 4A), the controller 130 receives a first high signal 152 via the first pin 130A. In the first closed state 114 (shown in FIG. 4B), the controller 130 receives a first low signal 154 via the first pin 130A. In the second open state 122 (shown in FIG. 4A), the controller 130 receives a second high signal 156 via the second pin 130B. In the second closed state 124 (shown in FIG. 4B), the controller 130 receives a second low signal 158 via the second pin 130B. Further, the controller 130 is configured to determine that the plurality of first conductive contacts 110 is in the first open state 112 upon receiving the first high signal 152 and the controller 130 is configured to determine that the plurality of first conductive contacts 110 is in the first closed state 114 upon receiving the first low signal 154. Similarly, the controller 130 is configured to determine that the plurality of second conductive contacts 120 is in the second open state 122 upon receiving the second high signal 156 and the controller 130 is configured to determine that the plurality of second conductive contacts 120 is in the second closed state 124 upon receiving the second low signal 158.

In some embodiments, the supply circuit 146 provides a second voltage 148 greater than about 0 V and less than about 5 V upon receiving the correct signal 138 from the controller 130. In some embodiments, the second voltage 148 is about 3.3 V. The second voltage 148 may enable measurement of the at least one electrical parameter 214 across the plurality of conductive pads 210. In some embodiments, the controller 130 is configured to measure the at least one electrical parameter 214 across the plurality of conductive pads 210 upon determining the correct condition 136 when the supply circuit 146 provides the second voltage 148.

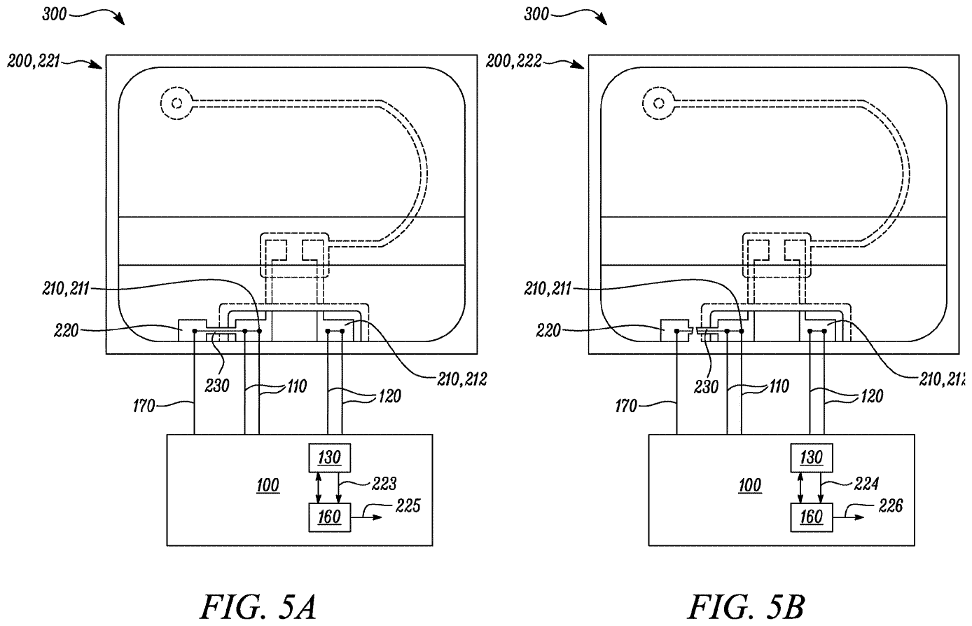

FIG. 5A is a schematic view of the sterilization indicator system 300, according to another embodiment of the present disclosure. Specifically, in the illustrated embodiment of FIG. 5A, the sensor device 200 is in an unused condition 221.

FIG. 5B is a schematic view of the sterilization indicator system 300 of FIG. 5B, according to an embodiment of the present disclosure. Specifically, in the illustrated embodiment of FIG. 5B, the sensor device 200 is in a used condition 222.

Referring to FIGS. 5A and 5B, in some embodiments, the reader device 100 further includes an additional conductive contact 170. In some embodiments, the controller 130 (shown in FIGS. 4A and 4B) is communicably coupled to the additional conductive contact 170.

Further, the sensor device 200 further includes an additional conductive pad 220 corresponding to the additional conductive contact 170. In some embodiments, the sensor device 200 further includes a conductive bridge 230 configured to electrically connect the additional conductive pad 220 to one of the plurality of conductive pads 210 upon insertion of the sensor device 200 within the slot 104 (shown in FIG. 2). In the illustrated embodiment of FIGS. 5A and 5B, the conductive bridge 230 is configured to conductively connect the additional conductive pad 220 to the first conductive pad 211. In such embodiments, the conductive bridge 230 is configured to electrically connect the additional conductive pad 220 to the first conductive pad 211 upon insertion of the sensor device 200 within the slot 104. However, in some other embodiments, the conductive bridge 230 may be configured to conductively connect the additional conductive pad 220 to the second conductive pad 212. In such embodiments, the conductive bridge 230 is configured to electrically connect the additional conductive pad 220 to the second conductive pad 212 upon insertion of the sensor device 200 within the slot 104.

The controller 130 is configured to determine, upon insertion of the sensor device 200 within the slot 104, the unused condition 221 (shown in FIG. 5A) if the additional conductive pad 220 is electrically connected to the one of the plurality of conductive pads 210. For example, in the illustrated example of FIG. 5A, the controller 130 is configured to determine, upon insertion of the sensor device 200 within the slot 104, the unused condition 221 if the additional conductive pad 220 is electrically connected to the first conductive pad 211. In some embodiments, the controller 130 is configured to generate an unused condition signal 223 upon determining the unused condition 221. In some embodiments, the alert circuit 160 is configured to generate an unused indication alert 225 upon receiving the unused condition signal 223 from the controller 130. In some embodiments, the unused indication alert 225 is at least one of a visual alert, an audio alert, and a haptic alert.

On the other hand, the controller 130 is configured to determine, upon insertion of the sensor device 200 within the slot 104, the used condition 222 (shown in FIG. 5B) if the additional conductive pad 220 is electrically disconnected from the one of the plurality of conductive pads 210. For example, in the illustrated example of FIG. 5B, the controller 130 is configured to determine, upon insertion of the sensor device 200 within the slot 104, the used condition 222 if the additional conductive pad 220 is electrically disconnected from the first conductive pad 211. In some embodiments, the controller 130 is configured to generate a used condition signal 224 upon determining the used condition 222. In some embodiments, the alert circuit 160 is configured to generate a used indication alert 226 upon receiving the used condition signal 224 from the controller 130. In some embodiments, the used indication alert 226 is at least one of a visual alert, an audio alert, and a haptic alert.

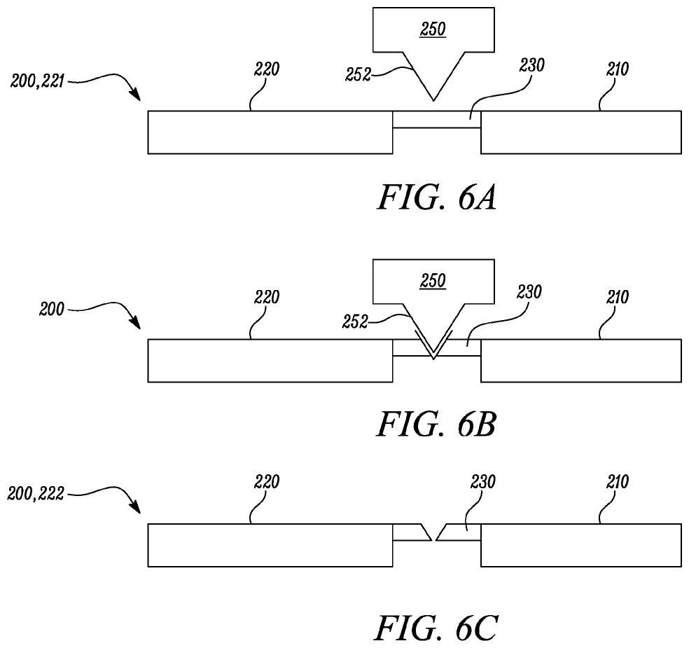

FIG. 6A is a schematic view of the sensor device 200 in the unused condition 221 and an accessory 250, according to an embodiment of the present disclosure. The accessory 250 includes a sharp portion 252. FIG. 6B is a schematic view of the sensor device 200 being used with the accessory 250, according to an embodiment of the present disclosure. FIG. 6C is a schematic view of the sensor device 200 in the used condition 222, according to an embodiment of the present disclosure.

Referring to FIGS. 6A-6C, in some embodiments, prior to insertion of the sensor device 200 within the slot 104 (shown in FIG. 2), the conductive bridge 230 breaks upon usage of the sensor device 200 with the accessory 250 having the sharp portion 252 corresponding to the conductive bridge 230. Therefore, in the used condition 222, the additional conductive pad 220 is electrically disconnected from the one of the plurality of conductive pads 210. For example, in the illustrated embodiment of FIG. 6C, in the used condition 222, the additional conductive pad 220 is electrically disconnected from the first conductive pad 211. Thus, the reader device 100 may be able to indicate if the sensor device 200 has been used with the accessory 250 prior to insertion of the sensor device 200 within the slot 104.

FIG. 7A is a schematic perspective view of the sterilization indicator system 300, according to an embodiment of the present disclosure. Specifically, in the illustrated embodiment of FIG. 7A, the sensor device 200 is in the correct position 204 with respect to the reader device 100. Therefore, the sterilization indicator system 300 in the correct condition 136. Further, the controller 130 (shown in FIG. 4B) measures the at least one electrical parameter 214 (shown in FIG. 4B) across the plurality of conductive pads 210 (shown in FIG. 4B) upon determining the correct condition 136. In the illustrated embodiment of FIG. 7A, the at least one electrical parameter 214 indicates that the efficacy of the sterilization process is adequate, i.e., the reader device 100 generates the pass alert 260. In the illustrated embodiment of FIG. 7A, the pass alert 260 is a visual alert. However, the pass alert 260 may be an audio alert or a haptic alert.

FIG. 7B is a schematic perspective view of the sterilization indicator system 300, according to an embodiment of the present disclosure. In the illustrated embodiment of FIG. 7B, the sensor device 200 is again in the correct position 204 with respect to the reader device 100. Therefore, the sterilization indicator system 300 in the correct condition 136. Further, the controller 130 (shown in FIG. 4B) measures the at least one electrical parameter 214 (shown in FIG. 4B) across the plurality of conductive pads 210 (shown in FIG. 4B) upon determining the correct condition 136. However, in the illustrated embodiment of FIG. 7B, the at least one electrical parameter 214 indicates that the efficacy of the sterilization process is not adequate, i.e., the reader device 100 generates the failure alert 270. In the illustrated embodiment of FIG. 7B, the failure alert 270 is a visual alert. However, the failure alert 270 may be an audio alert or a haptic alert.

FIG. 7C is a schematic perspective view of the sterilization indicator system 300, according to an embodiment of the present disclosure. In the illustrated embodiment of FIG. 7C, the sensor device 200 is in the incorrect position 202 with respect to the reader device 100. Specifically, the sensor device 200 is inserted backwards in the slot 104 (shown in FIG. 2). Therefore, the sterilization indicator system 300 in the error condition 132. The controller 130 (shown in FIG. 4A) therefore does not measure the at least one electrical parameter 214 (shown in FIG. 4A) across the plurality of conductive pads 210. Further, the controller 130 generates the error signal 134 (shown in FIG. 4A) upon determining the error condition 132 and the alert circuit 160 (shown in FIG. 4A) generates the error alert 162 (shown in FIG. 4A) upon receiving the error signal 134 from the controller 130. In the illustrated embodiment of FIG. 7C, the error alert 162 is a visual alert.

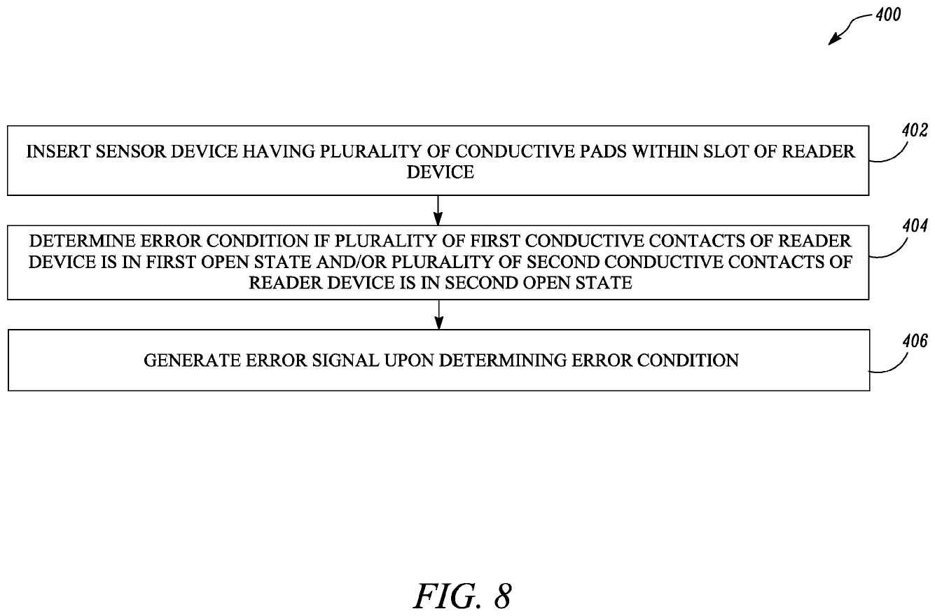

FIG. 8 is a flowchart illustrating a method 400, according to an embodiment of the present disclosure.

With reference to FIGS. 1 to 8, at step 402, the method 400 includes inserting the sensor device 200 having the plurality of conductive pads 210 within the slot 104 of the reader device 100. In some embodiments, the method 400 further includes determining that the sensor device 200 has been inserted within the slot 104 upon receiving the insertion signal 166 from the sensor 164.

As discussed above, the reader device 100 includes the plurality of first conductive contacts 110 disposed in the slot 104 and adjacent to each other. The plurality of first conductive contacts 110 is configured to electrically contact the corresponding one or more conductive pads 210 from the plurality of conductive pads 210 of the sensor device 200 upon insertion of the sensor device 200 within the slot 104. The plurality of first conductive contacts 110 includes the first open state 112 and the first closed state 114. In the first open state 112, the first conductive contacts 110 are electrically disconnected from each other. In the first closed state 114, the first conductive contacts 110 are electrically connected to each other.

Further, the reader device 100 includes the plurality of second conductive contacts 120 disposed in the slot 104 and adjacent to each other. The plurality of second conductive contacts 120 is spaced apart from the plurality of first conductive contacts 110. The plurality of second conductive contacts 120 is configured to electrically contact the corresponding one or more conductive pads 210 from the plurality of conductive pads 210 of the sensor device 200 upon insertion of the sensor device 200 within the slot 104. The plurality of second conductive contacts 120 includes the second open state 122 and the second closed state 124. In the second open state 122, the second conductive contacts 120 are electrically disconnected from each other. In the second closed state 124, the second conductive contacts 120 are electrically connected to each other.

At step 404, the method 400 includes determining the error condition 132 if the plurality of first conductive contacts 110 of the reader device 100 is in the first open state 112 and/or the plurality of second conductive contacts 120 of the reader device 100 is in the second open state 122. As discussed above, in the error condition 132, the sensor device 200 is in the incorrect position 202 with respect to the reader device 100.

At step 406, the method 400 includes generating the error signal 134 upon determining the error condition 132. In some embodiments, the method 400 further includes generating the error alert 162 upon receiving the error signal 134.

In some embodiments, the method 400 further includes determining, upon insertion of the sensor device 200 within the slot 104, the correct condition 136 if the plurality of first conductive contacts 110 is in the first closed state 114 and the plurality of second conductive contacts 120 is in the second closed state 124. As discussed above, in the correct condition 136, the sensor device 200 is in the correct position 204 with respect to the reader device 100. In some embodiments, the method 400 further includes generating the correct signal 138 upon determining the correct condition 136.

In some embodiments, the method 400 further includes measuring the at least one electrical parameter 214 across the plurality of conductive pads 210 upon determining the correct condition 136. As discussed above, the at least one electrical parameter 214 includes at least one of the voltage, the current flow, the resistance, and the impedance across the plurality of conductive pads 210.

In some embodiments, the method 400 further includes, upon insertion of the sensor device 200 within the slot 104, determining that the plurality of first conductive contacts 110 is in the first open state 112 upon receiving the first high signal 152. In some embodiments, the method 400 further includes, upon insertion of the sensor device 200 within the slot 104, determining that the plurality of first conductive contacts 110 is in the first closed state 114 upon receiving the first low signal 154. In some embodiments, the method 400 further includes, upon insertion of the sensor device 200 within the slot 104, determining that the plurality of second conductive contacts 120 is in the second open state 122 upon receiving the second high signal 156. In some embodiments, the method 400 further includes, upon insertion of the sensor device 200 within the slot 104, determining that the plurality of second conductive contacts 120 is in the second closed state 124 upon receiving the second low signal 158.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A reader device for use with a sensor device having a plurality of conductive pads, the reader device comprising:
a body defining a slot configured to at least partially receive the sensor device therein;
a plurality of first conductive contacts disposed in the slot and adjacent to each other, wherein the plurality of first conductive contacts is configured to electrically contact corresponding one or more conductive pads from the plurality of conductive pads of the sensor device upon insertion of the sensor device within the slot, wherein the plurality of first conductive contacts comprises a first open state and a first closed state, wherein, in the first open state, the first conductive contacts are electrically disconnected from each other, and wherein, in the first closed state, the first conductive contacts are electrically connected to each other;
a plurality of second conductive contacts disposed in the slot and adjacent to each other, wherein the plurality of second conductive contacts is spaced apart from the plurality of first conductive contacts, wherein the plurality of second conductive contacts is configured to electrically contact corresponding one or more conductive pads from the plurality of conductive pads of the sensor device upon insertion of the sensor device within the slot, wherein the plurality of second conductive contacts comprises a second open state and a second closed state, wherein, in the second open state, the second conductive contacts are electrically disconnected from each other, and wherein, in the second closed state, the second conductive contacts are electrically connected to each other; and
a controller communicably coupled to at least one of the plurality of first conductive contacts and at least one of the plurality of second conductive contacts, wherein the controller is configured to:
determine, upon insertion of the sensor device within the slot, an error condition if the plurality of first conductive contacts is in the first open state or the plurality of second conductive contacts is in the second open state, wherein, in the error condition, the sensor device is in an incorrect position with respect to the reader device; and
generate an error signal upon determining the error condition.

2. The reader device of claim 1, wherein the controller is further configured to determine, upon insertion of the sensor device within the slot, a correct condition if the plurality of first conductive contacts is in the first closed state and the plurality of second conductive contacts is in the second closed state, and wherein, in the correct condition, the sensor device is in a correct position with respect to the reader device.

3. The reader device of claim 2, wherein the controller is further configured to generate a correct signal upon determining the correct condition.

4. The reader device of claim 2, wherein the controller is further configured to measure at least one electrical parameter across the plurality of conductive pads upon determining the correct condition, and wherein the at least one electrical parameter is indicative of an efficacy of a sterilization process.

5. The reader device of claim 4, wherein the at least one electrical parameter comprises at least one of a voltage, a current flow, a resistance, and an impedance across the plurality of conductive pads.

6. The reader device of claim 1, wherein:
the plurality of first conductive contacts is spaced apart from each other by a first distance;
the plurality of second conductive contacts is spaced apart from each other by a second distance; and
wherein each of the first distance and the second distance is less than or equal to about 5 millimeters (mm) and greater than or equal to about 1 mm.

7. The reader device of claim 3, wherein:
the plurality of first conductive contacts comprises a pair of first conductive contacts spaced apart from each other;
the plurality of second conductive contacts comprises a pair of second conductive contacts spaced apart from each other;
the pair of first conductive contacts is configured to electrically contact a first conductive pad from the plurality of conductive pads of the sensor device upon insertion of the sensor device within the slot; and
the pair of second conductive contacts is configured to electrically contact a second conductive pad from the plurality of conductive pads of the sensor device upon insertion of the sensor device within the slot.

8. The reader device of claim 7, further comprising:
a voltage source;
a first pullup resistor;
a second pullup resistor; and
a supply circuit electrically connected to the voltage source,
wherein the controller is communicably coupled to the supply circuit and comprises a first pin and a second pin, and wherein:
one of the pair of first conductive contacts is electrically connected to the first pin of the controller and the voltage source, and the first pullup resistor is electrically disposed between the one of the pair of first conductive contacts and the voltage source;
the other of the pair of first conductive contacts is electrically connected to electrical ground;
one of the pair of second conductive contacts is electrically connected to the second pin of the controller and the voltage source, and the second pullup resistor is electrically disposed between the one of the pair of first conductive contacts and the voltage source;
the other of the pair of second conductive contacts is electrically connected to the supply circuit, the supply circuit provides a first voltage equal to about 0 volts (V) when the sensor device is inserted within the slot;
in the first open state, the controller receives a first high signal via the first pin;
in the first closed state, the controller receives a first low signal via the first pin;
in the second open state, the controller receives a second high signal via the second pin;
in the second closed state, the controller receives a second low signal via the second pin;
the controller is configured to determine that the plurality of first conductive contacts is in the first open state upon receiving the first high signal;
the controller is configured to determine that the plurality of first conductive contacts is in the first closed state upon receiving the first low signal;
the controller is configured to determine that the plurality of second conductive contacts is in the second open state upon receiving the second high signal; and
the controller is configured to determine that the plurality of second conductive contacts is in the second closed state upon receiving the second low signal.

9. The reader device of claim 8, wherein the supply circuit provides a second voltage greater than about 0 V and less than about 5 V upon receiving the correct signal from the controller.

10. The reader device of claim 1, further comprising an alert circuit communicably coupled to the controller, wherein the alert circuit is configured to generate an error alert upon receiving the error signal from the controller, and wherein the error alert is at least one of a visual alert, an audio alert, and a haptic alert.

11. The reader device of claim 1, further comprising a sensor communicably coupled to the controller and configured to generate an insertion signal upon sensing the insertion of the sensor device within the slot, wherein the controller is further configured to determine that the sensor device has been inserted within the slot upon receiving the insertion signal from the sensor.

12. A sterilization indicator system comprising:
a sensor device comprising a plurality of conductive pads; and
the reader device of claim 1, wherein, upon insertion of the sensor device within the slot, the plurality of first conductive contacts is configured to electrically contact corresponding one or more first conductive pads from the plurality of conductive pads of the sensor device and the plurality of second conductive contacts is configured to electrically contact corresponding one or more second conductive pads from the plurality of conductive pads of the sensor device.

13. The sterilization indicator system of claim 12, wherein:
the reader device further comprises an additional conductive contact; and
the sensor device further comprises:
an additional conductive pad corresponding to the additional conductive contact;
a conductive bridge configured to electrically connect the additional conductive pad to one of the plurality of conductive pads upon insertion of the sensor device within the slot; and
prior to insertion of the sensor device within the slot, the conductive bridge breaks upon usage of the sensor device with an accessory having a sharp portion corresponding to the conductive bridge.

14. The sterilization indicator system of claim 13, wherein the controller is communicably coupled to the additional conductive contact, and wherein the controller is configured to determine, upon insertion of the sensor device within the slot, a used condition if the additional conductive pad is electrically disconnected from the one of the plurality of conductive pads.

15. The sterilization indicator system of claim 14, wherein the controller is configured to generate a used condition signal upon determining the used condition.

16. The sterilization indicator system of claim 15, wherein the reader device further comprises an alert circuit communicably coupled to the controller, wherein the alert circuit is configured to generate a used indication alert upon receiving the used condition signal from the controller, and wherein the used indication alert is at least one of a visual alert, an audio alert, and a haptic alert.

17. A method comprising:
inserting a sensor device having a plurality of conductive pads within a slot of a reader device, wherein the reader device comprises:
a plurality of first conductive contacts disposed in the slot and adjacent to each other; wherein the plurality of first conductive contacts is configured to electrically contact corresponding one or more conductive pads from the plurality of conductive pads of the sensor device upon insertion of the sensor device within the slot, wherein the plurality of first conductive contacts comprises a first open state and a first closed state, wherein, in the first open state, the first conductive contacts are electrically disconnected from each other, and wherein, in the first closed state, the first conductive contacts are electrically connected to each other; and
a plurality of second conductive contacts disposed in the slot and adjacent to each other, wherein the plurality of second conductive contacts is spaced apart from the plurality of first conductive contacts, wherein the plurality of second conductive contacts is configured to electrically contact corresponding one or more conductive pads from the plurality of conductive pads of the sensor device upon insertion of the sensor device within the slot, wherein the plurality of second conductive contacts comprises a second open state and a second closed state, wherein, in the second open state, the second conductive contacts are electrically disconnected from each other, and wherein, in the second closed state, the second conductive contacts are electrically connected to each other;
determining an error condition if the plurality of first conductive contacts of the reader device is in the first open state and/or the plurality of second conductive contacts of the reader device is in the second open state, wherein, in the error condition, the sensor device is in an incorrect position with respect to the reader device; and
generating an error signal upon determining the error condition.

18. The method of claim 17, further comprising determining, upon insertion of the sensor device within the slot, a correct condition if the plurality of first conductive contacts is in the first closed state and the plurality of second conductive contacts is in the second closed state, wherein, in the correct condition, the sensor device is in a correct position with respect to the reader device.

19. The method of claim 18, further comprising generating a correct signal upon determining the correct condition.

20. The method of claim 18, further comprising measuring at least one electrical parameter across the plurality of conductive pads upon determining the correct condition.

21. The method of claim 20, wherein the at least one electrical parameter comprises at least one of a voltage, a current flow, a resistance, and an impedance across the plurality of conductive pads.

22. The method of claim 17, further comprising, upon insertion of the sensor device within the slot:
determining that the plurality of first conductive contacts is in the first open state upon receiving a first high signal;
determining that the plurality of first conductive contacts is in the first closed state upon receiving a first low signal;
determining that the plurality of second conductive contacts is in the second open state upon receiving a second high signal; and
determining that the plurality of second conductive contacts is in the second closed state upon receiving a second low signal.

23. The method of claim 17, further comprising generating an error alert upon receiving the error signal, wherein the error alert is at least one of a visual alert, an audio alert, and a haptic alert.

24. The method of claim 17, further comprising determining that the sensor device has been inserted within the slot upon receiving an insertion signal from a sensor.
